(19) United States
(12) Patent Application Publication
(10) Pub. No.: US 2025/0352477 A1
(43) Pub. Date: Nov. 20, 2025

(54) FORMULATIONS AND ORAL DOSAGE FORMS OF LIPOPHILIC AGENTS

(71) Applicant: CANNBIOREX PHARMA LTD, London Greater London (GB)

(72) Inventors: Abraham Jackob DOMB, Jerusalem (IL); Marc FELDMANN, London Greater London (GB)

(73) Assignee: CANNBIOREX PHARMA LTD, London Greater London (GB)

(21) Appl. No.: 18/871,183

(22) PCT Filed: Jun. 5, 2023
(86) PCT No.: PCT/IL2023/050574

§ 371 Date: Dec. 3, 2024

Related U.S. Application Data
(60) Provisional application No. 63/365,884, filed on Jun. 6, 2022.

(51) Int. Cl. A61K 9/20; A61K 31/00; A61K 47/32; A61K 47/38

(52) U.S. Cl. CPC A61K 9/2077; A61K 31/658; A61K 47/32; A61K 47/38

(57) ABSTRACT

The invention includes improved pharmaceutical formulations of lipophilic actives, and specifically solid and semi-solid forms and oral dosage forms of such formulations that exhibit improved properties of controlled or modifiable release of actives and actives bioavailability upon dissolution in aqueous conditions at body temperature.

### TECHNOLOGICAL FIELD

The invention pertains to the field of pharmaceutical formulations of lipophilic actives, and specifically to solid and semi-solid forms and oral dosage forms of such formulations that exhibit improved properties of controlled or modifiable release of actives and actives' bioavailability upon dissolution in aqueous conditions at body temperature.

### BACKGROUND

Oral bioavailability of drugs generally depends on the ability of the drug formulation or the formulated active to cross the gastrointestinal (GI) mucosa. Hydrophobic or lipophilic actives are generally poorly absorbed the GI tract due to the poor solubility and/or the tendency to disperse as nanostructures in aqueous GI milieu. Another important factor that can contribute to low bioavailability is enzymatic degradation of the drug, which typically occurs in the liver and the gut lumen or epithelia, i.e., the first and the second pass metabolism. This enzymatic component strongly depends on the chemical structure of the drug.

Cannabinoids, for example, are problematic by both these parameters: for being highly lipophilic and therefore practically insoluble in aqueous milieu, and for being subjected to intensive first and second pass metabolism (90%), and thus are generally considered to have an exceptionally poor oral bioavailability.

Therefore, producing successful oral formulations of lipophilic drugs, and specifically cannabinoids, remains one of the biggest challenges of pharma industry, especially in view that most of the drugs in the early stages of development have high lipophilicity. Certain examples of solid oral formulations of lipophilic actives, and cannabinoids in particular, were disclosed in WO 2008/033024 and US 2015/0132400, using micro-granulate material such as lactose. These however were not formulations producing cannabinoid loaded nanoparticles, and therefore did not have the advantages of improved solubility, absorption and oral bioavailability that are associated with nanoparticulate formulations.

Previous studies and publications by the inventor described liquid formulations of lipophilic actives, such as tetrahydrocannabinol (THC), cannabidiol (CBD), cyclosporin, paclitaxel and amphotericin B, which were dissolved in an anhydrous liquid composed of surfactants, organic solvents and low melting lipids. In the context of oral use, this type of formulation generally requires loading into soft gelatin capsules.

More generally, the liquid formulations are known for several typical limitations: (1) limited stability over time due to solvent evaporation, (2) interaction of the lipophilic actives or other ingredients with soft gelatin capsule, (3) precipitation and/or increase in particles size upon dispersion or contact with aqueous media, and other considerations since soft gelatin capsules are known to be relatively expensive and have limited shelf life and high batch-to-batch variability. Importantly, liquid formulations do not display controlled release capabilities and are generally limited to immediate release of actives.

### GENERAL DESCRIPTION

In most general terms, the invention provides means of producing solid compositions of poorly water soluble or lipophilic actives or drugs. One advantage of such compositions is the ability to prolong shelf life and durability of the incorporated lipophilic actives, and to avoid the need for soft gelatin capsules. An important advantage of the compositions in this application is that they are highly homogenous as solids, with no evidence of crystallization, yet they easily reach a complete dissolution or dispersion in an aqueous milieu. Homogeneity per se is important since it makes the compositions easily adaptable for producing various oral and topical dosage forms and to avoid batch-to-batch variability. In addition, when contacting or dissolving the compositions in an aqueous medium (e.g., oral or gastric fluids or moisture of the skin), the compositions spontaneously form drug containing nanoparticles. This feature is particularly important as it is directly related to improved drug solubility and dispersibility in body fluids and improved drug transport across biological membranes, and drug protection against liver and gut metabolism. Taken together, this feature is reflected in two main hallmarks of the present compositions, being improved drug absorption and drug bioavailability. Another important distinguishing feature of the present compositions is in providing controlled release (immediate, sustained, prolonged release) of the lipophilic actives, the ability to modify the extent of release by varying the type and the proportions between non-active components.

The invention is based on surprising findings by the inventor that liquid pro-nano-lipid formulations (LPNFs) of lipophilic drugs can be adsorbed onto particles of a porous inorganic polymer to form solid formulations without compromising their ability to form drug containing nanoparticles in aqueous medium and with all the ensuing benefits of improved solubility and improved bioavailability. Studies in a rat model revealed that these solid compositions can be further enhanced by the inclusion of certain non-active ingredients to provide controlled release of the drug containing nanoparticles and were also compatible with different lipophilic drugs.

These studies have led to the notion that the inventor has succeeded to produce a highly adaptable platform for the design and development of compositions with improved and controlled drug delivery, which has led to the compositions in this application.

More specifically, the invention provides solid or semi-solid compositions comprising a lipophilic active and a mixture of non-active ingredients, composed of one or more lipids, amphiphilic solvents and surfactants (emulsifiers) in solid or semi-solid states. The inventor has shown that while liquid lipids and surfactants yield liquid pro-nano-lipid formulations (LPNFs), the solid or semi-solid types of these ingredients yield solid or semi solid pro-nano-lipid formulations (SPNFs). Both LPNFs and SPNFs can be successfully adsorbed into a wide range of adsorbents and compressed into tablets or other pharmaceutically advantageous forms. But SPNF can produce solid and semi-solid formulations without the solid adsorbent particles.

Examples of lipids that can be incorporated into the compositions of the invention, LPNFs and SPNFs, are fatty acids, fatty acid esters, fatty amines, fatty amides and fatty alcohols. SPNFs can further use waxes that are esters of fatty alcohols and fatty acids, and PEG conjugates with fatty acids. Examples of suitable surfactants are Tweens, Spans, Labrasoles, Labrafils. Examples of suitable adsorbents are microcrystalline celluloses, chemically modified celluloses or starch, polyacrylates, chalk, charcoal, porous silica and synthetic allyl polymers. Successful solid compositions comprising some of these materials have been presently exemplified.

An important distinctive physical feature of the present compositions is their relatively high melting temperature, at least about 30° C. and surprising homogeneity at melting point. The melting profiles of some solid compositions derived from SPNF-blank or SPNF-drug revealed a single endotherm by differential scanning calorimetry (DSC), with no evidence of crystallization or sedimentation of trace materials. This feature is highly important to be to meet the requirements of uniformity of weight and drug dosage, and further prolonged stability.

As has been noted, an important advantage of the present compositions is that upon contact with water or a water-based medium, they spontaneously release drug containing nanoparticles of a relatively constant size, with a diameter of up to about 500 nm, or even below 300-200 nm. The nanoparticles assist the availability and the dispersibility of the drug into the water-based medium or system such as the GI, blood, a target tissue of the organism. The rate of release can be modified by the incorporation of various non-active components that affect the availability and dispersibility of the nanoparticles and the encapsulated drug. Comparisons with specific combinations of such non-active components that attenuate or provide prolonged release active have presently been exemplified.

The compositions of the invention can be provided in a variety of forms, such as powders, solid molten forms, compressed tablets and further included into hard gelatin capsules and devices of films to serve different types of administration and specific clinical applications. It is projected that the compositions or the formulations of the invention can be compatible with various types of lipophilic drugs (as per the definitions of FDA/EMA) and also other types of other lipophilic actives, such as lipophilic nutraceuticals, foods, food additives and lipophilic cosmetics (as per the definition of GRAS). They can further serve agricultural purposes in providing highly practical solid compositions of lipophilic herbicides, pesticides, etc. Importantly, the compositions of the invention are modifiable by incorporation of various non-active ingredients to provide different types of active's release, including immediate, sustained, prolonged release and potentially targeted release in the future, thus providing a highly valuable and adaptable core technology that can be implemented into the pharma, nutraceutical, food and cosmetic industries and agriculture.

### BRIEF DESCRIPTION OF DRAWINGS

To better understand the subject matter and to exemplify how it may be carried out in practice, specific embodiments of the invention will now be described by way of examples and drawings, which are not meant to be limiting.

FIG. 1 illustrates the main steps in the process of preparation of solid CBD compositions derived from LPNF-CBD adsorbed into silica powder (Neusilin) at 0.5 ratio w/v, starting from the liquid form (1a), through the adsorbed powder form (1b-1c) and ending with the compressed tablet from (1d).

FIG. 2 shows pharmacokinetic (PK) profiles (log scale) of various types of compositions as revealed in a freely moving rat model, including animals receiving: (1) CBD in propylene glycol:ethanol:water solution (control), (2) LPNF-CBD (liquid CBD PNL), (3) LPNF-CBD Polypore (CBD PNL adsorbed polypore) and (4) LPNF-CBD Neusilin (CBD neusilin powder). Compositions (2) to (5) were dispersed in water and administered orally, composition (1) was administered IV. LPNF-CBD Neusilin exhibited a better PK performance compared to the other compositions that was supported by calculations of AUC, showing 1.5-fold difference from CBD in solution (statistically significant). Importantly, it did not interfere with the original LPNF properties.

FIG. 3 illustrates the properties of several solid LPNF-blank compositions using various types of solid absorbents (Syloid, Neusilin, Eudragit (acrylic copolymer), Microcrystalline Cellulose (MCC), Ethyl Cellulose (EC), Mannitol, Carbopol, Charcoal, Wood Charcoal and Starch). The best results in terms of adsorpsion capacity, dryness and texture were achieved with Syloid, Neusilin, MCC, EC and Charcoal.

FIG. 4 shows CBD release from several solid LPNF-CBD compositions at neutral pH (200 mg powders in 40 ml PBS pH=7), using solid absorbents with the best absorption capacity (Syloid, Neusilin, MCC, EC, Charcoal). The highest release was observed with compositions using Neusilin or MCC.

FIG. 5 shows CBD release from tablets produced with various solid absorbents that were subjected to GI mimicking conditions (gastric solution (pH˜2) for 2 h and intestinal solution (pH˜7) for additional 2-6 h). Tablets using MCC as the only absorbent (MAE-3-19-A) had the fastest CBD release with up to 60% CBD at 2 h and 95% at 8 h. The second best was tablets using MCC and lactose (MAE-3-24-B) with up to 75% release at 8 h time. Tablets, with lower lactose and/or no MCC or with HPMC or Neusilin, had lower CBD release.

FIG. 6 illustrates the appearance of Neusilin and MCC tablets at (A and B, respectively) at time 0 and 6 h in this experiment (FIG. 5). Neusilin tablets generally had lower dissolution and CBD release and were also less advantageous in terms of particle size.

FIG. 7 illustrates the appearance of SPNF composition constructed from IMWITOR® 900 K, Tween 80, Kollipor RH 40, PEG 1000, Labrafil® M 1944 CS with and without CBD (MA-9-31-A). Images show the consistencies of the SPNF-blank (a), SPNL-CBD (b) and SPNF-blank water dispersion (c), suggesting a complete dissolution.

FIG. 8 shows particle size analysis of the water dispersions of the same preparations (FIG. 7) by dynamic light scattering (DLS). Both SPNF-blank (left) and SPNF-CBD (right) exhibited single peak profiles with average particle size in the range of <200 nm, specifically 177.8±5.1 nm and 139.7±3.7 nm, respectively. SPNF-CBD had lower particles size than the blank preparation.

FIG. 9 shows melting point analysis of selected SPNF preparations, SPNF-blank, SPNF-CBD and SPNF-Curcumin, by differential scanning calorimetry (DSC). All samples exhibited melting profiles with a single endotherm (approx. 60° C.), suggesting homogeneity, no crystallization or sedimentation and a complete dissolution of the incorporated active.

FIG. 10 shows CBD release from a prototype powder LPNF-CBD (5% CBD w/w) absorbed on MCC and incubated in GI mimicking conditions (FIG. 5), showing a prolonged release profile with increasing CBD release for up to 4 h and reaching plateau after 6 h.

FIG. 11 shows pharmacokinetic (PK) profiles of the powder LPNF-CBD and SPNF-CBD formulations vs. liquid control formulation after oral administration in equal effective doses to rats (15 mg CBD/kg). All formulations, liquid control formulation (●), LPNF-CBD) (▪) and SPNF-CBD (Δ), exhibited the capabilities of controlled release for the duration of the experiment (up to 8 h).

FIG. 12 shows the same data on semilogarithmic scale, suggesting similar prolonged release behavior in all formulations. Subsequent more detailed analysis of the PK parameters suggested that the SPNF-CBD formulation had the best PK performance in terms of AUC, Cmax and Absolute bioavailability.

### DETAILED DESCRIPTION OF EMBODIMENTS

It is one of the main objectives of the invention to provide successful and readily usable formulations or compositions with lipophilic activities. In the broadest terms, the compositions of the invention are solid or semi-solid compositions comprising at least one lipophilic active and a solid or semi-solid mixture comprising at least one material from the following groups:
a surfactant or an emulsifier,
a lipid
an amphiphilic solvent

In some embodiments the lipid can be selected from the group of fatty acids, fatty acid esters, fatty amines, fatty amides and fatty alcohols,

In some embodiments the compositions can further comprise at least one absorbent.

One of the distinguishing features of the present compositions is in having a melting point at a temperature of at least about 30° C., which is consistent with their consistency at room temperature.

In some embodiments the compositions can have a melting temperature in the range between 30° C.-40° C., 40° C.-50° C., 50° C.-60° C., 60° C.-70° C., 70° C.-80° C., 80° C.-90° C., 90° C.-100° C. and more.

An important feature of the present compositions is that they can be readily dissolved or dispersed in an aqueous solution in producing nanoparticles with a size of less than about 500 nm, or less than about 500 nm, 400 nm, 300 nm, 200 nm.

In some embodiments the compositions can have particle size in the range between about 500-400 nm, 400-300 nm, 300-200 nm and 200-100 nm.

This feature is directly responsible for another important characteristics of the present compositions, being the ability to provide controlled release and improved bioavailability to the entrapped lipophilic actives. This feature is revealed upon contact or dispersion of the compositions in an aqueous medium. In the medical context, it is revealed upon dissolution or dispersion of the compositions in body fluids.

In some embodiments the body fluids can be oral, gastric fluids, serum or blood, or moisture of the skin. The term “body fluids” is applied herein to humans and other mammals.

In some embodiments the controlled release can comprise immediate release of the actives.

In some embodiments the controlled release can comprise sustained release of the actives.

In some embodiments the controlled release can comprise prolonged release of the actives.

Another distinctive feature of the present compositions is revealed upon melting analysis, showing a homogenous consistency with no evidence of crystallization or sedimentation. This feature is directly responsible for the manageability and prolonged shelf life, and a series of other advantages of the present compositions.

In some embodiments the melting analysis can be performed by differential scanning calorimetry (DSC), whereby the compositions are exhibiting a characteristic single endotherm.

In some embodiments the single the single endotherm can be exhibited at a temperature in the range between about 40° C. and about 80° C., or more specifically in the range between about 40° C.-50° C., 50° C.-60° C., 60° C.-70° C., 70° C.-80° C.

In some embodiments the single endotherm can be exhibited at a temperature in the range of 30° C.-40° C., 40° C.-50° C., 50° C.-60° C., 60° C.-70° C., 70° C.-80° C., 80° C.-90° C., 90° C.-100° C. or more.

In some embodiments the single endotherm can be exhibited at about 60° C.

Regarding the main constituents of these compositions:

In some embodiments the lipid component can be selected from the group of mono-, di- and triglycerides, fatty alcohols and waxes.

In some embodiments the waxes can be selected from the group of esters of fatty alcohols, fatty acids and polyethylene glycol (PEG) PEG conjugates with fatty acids.

In some embodiments the surfactant or emulsifier can be selected from the group of Tweens, Spans, Labrasoles, Labrafils.

In some embodiments the amphiphilic solvent can be selected from the group of short alcohols, propylene glycol, glycerol, esters like ethyl lactate.

In some embodiments the adsorbents can be selected from the group of microcrystalline celluloses, chemically modified celluloses or starch, polyacrylates, chalk, charcoal, porous silica and synthetic allyl polymers.

In some embodiments the polyacrylates can be selected from the group of copolymers of acrylic acid and methyl methacrylate.

In some embodiments the synthetic allyl polymer can be Polypore.

In some embodiments the at least one adsorbent is microcrystalline cellulose.

More generally, the presently proposed formulation approach pertains to many types of lipophilic actives and other lipophilic substances, which can be generally defined as a substance having a logP>1. The lipophilic actives can be selected for medical purposes, agricultural purposes, substances which make part of foods and food additives (preservatives, stabilizers, aroma and flavour substances, etc.).

In some embodiments the actives can be selected from the group of lipophilic therapeutic and nutraceutical agents

In some embodiments the lipophilic active can be a therapeutic agent selected from the group of Cannabinoids, a mixture of Cannabinoids, Cyclosporin, Paclitaxel and Amphotericin B, which are known for their lipophilicity and difficulties to find suitable formulations.

The term “cannabinoids” encompasses herein all types of known cannabinoids, including natural, synthetic and modified natural cannabinoids, and mixes thereof. It further encompasses all the main cannabinoids derived from various types of Cannabis plants, including but not limited to Tetrahydrocannabinol (THC), Cannabidiol (CBD), Cannabinol (CBN), Cannabigerol (CBG), Cannabichromene (CBC), Cannabicyclol (CBL), Cannabivarin, (CBV), Tetrahydrocannabivarin (THCV), Cannabidivarin (CBDV), Cannabichromevarin (CBCV), Cannabigerovarin (CBGV), Cannabigerol Monomethyl Ether (CBGM), as well as derivatives, precursors and form thereof. It further encompasses any extracts of Cannabis plants, including but not limited to extracts of C. Sativa, C. Indica and C. Ruderalis.

In some embodiments the Cannabinoid can be Tetrahydrocannabinol (THC) or Cannabidiol (CBD), or a derivative, a precursor, an acid form, or a mixture thereof.

In some embodiments the lipophilic active can be a nutraceutical agent selected from the group of lipophilic Vitamins, Carotenoids, Lycopene, Lutein, Curcumin, Resveratrol and Coenzyme Q.

In the medical context, the compositions of the invention can serve multiple purposes, and to that end can be adapted for various modes of delivery, including oral, buccal, ophthalmic, topical administrations or subcutaneous, intramuscular, intravenous and intraocular injections and inhalation, with and without assisting devices.

For the injectable forms, the compositions can be dispersed into a nanoparticulate liquid suitable for SC, IM or IV injections. The compositions can be loaded in water soluble porous particles such as sugar microparticles to form solid water-dispersible formulation that can be delivered topically in the form of eye drops or nasal spray.

One of the most attractive implementations of the present compositions is as oral compositions.

In some embodiments the composition can be used in the manufacture of agricultural agents.

In some embodiments the composition can be used in the manufacture of foods and food additives (food preservatives, stabilizers, aroma and flavour substances, etc.) To that end, the compositions carrying various valuable actives can be dispersed in beverages and foods, or incorporated into gummies,

In some embodiments the compositions can be incorporated into fast dissolving films or cosmetic formulations.

Thus, it another objective of the invention to provide compositions for specific uses in various industries.

In some embodiments the invention can provide pharmaceutical or nutraceutical compositions comprising one or more of the above-mentioned compositions.

In some embodiments such pharmaceutical and nutraceutical compositions can further comprise a pharmaceutically acceptable carrier or excipient.

In some embodiments the invention can provide oral dosage forms comprising one or more of the above-mentioned compositions.

In some embodiments the invention can provide topical formulations comprising one or more of the above-mentioned compositions.

In some embodiments the invention can provide topical devices comprising one or more of the above-mentioned compositions. The term “topical device” encompasses herein topical films, skin patches and other devices for topical drug delivery.

In some embodiments the invention can provide ophthalmic formulations comprising one or more of the above-mentioned compositions.

In some embodiments the invention can provide cosmetic formulations comprising one or more of the above-mentioned compositions.

In some embodiments the invention can provide foods, food supplements and food additives comprising one or more of the above-mentioned compositions.

For another point of view, the compositions of the invention can be articulated in terms of compositions for improved delivery of lipophilic actives to mammals (human and other).

In some embodiments the delivery can comprise oral, buccal, ophthalmic, topical delivery or subcutaneous, intramuscular, intravenous and intraocular injections or inhalation of the compositions.

In some embodiments the improved delivery can comprise improved solubility of the lipophilic actives in body fluids.

In some embodiments the improved delivery can comprise improved adsorption of the lipophilic actives in the GI tract, blood and/or body tissues.

In some embodiments the improved delivery can comprise improved bioavailability of the lipophilic actives in the GI tract, blood and/or body tissues.

In some embodiments the improved delivery can further comprise immediate delivery of the lipophilic actives into the GI tract, blood and/or body tissues.

In some embodiments the improved delivery can further comprise sustained delivery of the lipophilic actives into the GI tract, blood and/or body tissues.

In some embodiments the improved delivery can further comprise prolonged delivery of the lipophilic actives into the GI tract, blood and/or body tissues.

For yet another point of view, the compositions of the invention can be used for treating various disorders and clinical and non-clinical conditions in mammals and humans.

In some embodiments the compositions of the invention can be used for treating disorders or conditions that are treatable by cannabinoids or combination thereof.

This aspect can be further articulated in terms of methods of treating disorders or conditions in mammals and humans. In the main, the methods of the invention involve administering one or more of the above-mentioned compositions to the mammal, wherein the administering can be oral, buccal, ophthalmic, topical delivery or subcutaneous, intramuscular, intravenous and intraocular injections or inhalation.

In some embodiments the methods of the invention can be applied to disorders or conditions that are treatable by cannabinoids or combination thereof.

This aspect can be further articulated in terms of the use of the above-mentioned compositions in the manufacture of medicaments for treating various types of disorders or conditions in mammals, as well as in the manufacture of agricultural and cosmetic agents.

### DETAILED DESCRIPTION OF APPLICATIONS

One of the most urgently needed applications of oral slow-release CBD formulations is in pain and inflammatory disease. A pharmaceutically preferable form, by manufacturers and consumers alike, is a tablet or a pill. Pain is a major health problem that remains a challenge to patients and practitioners all over the world, it affects hundreds of millions of individuals. While there are medications which can control pain effectively their use has some major problems, including a major risk of addiction with opioids as well as their serious side effects, especially in elderly. This has led to significant changes in clinical practice, from general practice of using large extremely hazardous amounts of opioids after an operation to almost no pain relief to avoid the risks of addiction and overdosing.

Opioid sparing is an important goal. While providing the most effective pain relief, they have dramatic side effects and a high risk of addition. Codeine is a less powerful opioid, with a lower potency, a fewer side effects and a lesser risk of addiction, can be effective in many conditions. Non-steroidal anti-inflammatory drugs (NSAIDs) are useful for short term pain, such as headache, dental pain, etc., but are much less effective for serious pain relief and may have side effects with chronic use, including kidney disease and gut ulcerations and bleeding. Paracetamol has less side effects than NSAIDs and can be effective for less severe pain. Neuropathic pain can be treated with different types of medicines, such as amitriptyline, duloxetine, pregabalin and gabapentin.

Ultimately with all current treatments, pain and chronic pain particular remains a major unmet medical need, especially for non-addictive analgesia. The problem of pain can be addressed, at least in part, by cannabinoids. Cannabinoids or medical marijuana are used by a very large number of individuals, predominantly fore chronic pain. However, they are still not sufficiently tested and absorbed preparations of cannabinoids which can be used effectively and reproducibly for pain indication are still lacking. There is a major need for such well absorbed cannabinoid preparations which are stable, manageable and readily applicable, and which could be well characterized in clinical trials.

Inflammation is an important component of many diseases, acute and chronic. A wide spectrum of very powerful anti-inflammatory drugs has been developed in recent years such as anti-cytokine monoclonal antibodies for TNF and IL-6 receptor that are used in the contexts of rheumatoid arthritis (RA) and inflammatory bowel disease (IBD), for example. However, they still suffer from significant disadvantages, including the need for repeated injections, side effects and incurred costs. The orally available anti-inflammatories, such as JAK inhibitors, suffer from even bigger disadvantages in these respects.

Thus, the development of improved orally available CBD formulations for treating chronic pain holds great potential. Much is known of the neuro-psychological effects of marijuana. In the medical context, however, psychotic effects are undesirable and should be separated from other medically beneficial effects. A breakthrough came with the discovery that the effects of CBD could be delineated from THC (Karniol and Carlini 1972, 1973), and further that CBD can attenuate the effects of THC when co-administrated (Zuardi, Hallak, and Crippa 2012; Zuardi and Karniol 1983). This significant finding led to the development of Sativex®, CBD and THC in equal parts, and its approval in many countries for the treatment of pain and spasticity in multiple sclerosis. Importantly, when taken alone CBD exhibits a broad spectrum of activities independent of other constituents of marijuana. Its anti-epileptic properties are well described, which have led to the approval of oral CBD Epidiolex® for the treatment of certain types of juvenile epilepsy.

### CBD, Pain, Inflammation and Anxiety

There is an accumulating large body of evidence that CBD has anxiolytic activity, this in addition to the well described anti-inflammatory capabilities (Burstein 2015; Crippa et al. 2009; Nichols and Kaplan 2020). Although the mechanism of this effect is still unclear, it positions CBD as promising candidate for the treatment of pain and chronic pain, particularly in the context of inflammatory diseases, such as RA, IBD, fibromyalgia, and others, which are often linked to long term anxiety.

### Direct Effects on Pain

There are a number of studies reporting on positive responses of patients with pain to CBD, which were further corroborated by scientific models (Mlost, Bryk, and Starowicz 2020). To date, clinical studies suggest that analgesia is achieved, for the most part, when CBD is co-administered with Δ-9-THC, such as in Sativex®. Reduced pain is one of the recorded benefits of Sativex in multiple sclerosis (MS), together with improved sleep and reduced insomnia and fatigue (Hoggart et al. 2015; Johnson et al. 2010; Serpell et al. 2014). Positive effects of CBD on mood, anxiety and sleep are likely to contribute to its ability to modulate perceived pain (Crippa et al. 2009). In addition, CBD was shown to reduce chronic pain after kidney transplantation and in the lower extremities of patients with peripheral neuropathy (Cunetti et al. 2018; Xu et al. 2020). CBD is known to signal via G-protein coupled receptors (GPCRs), including serotoninergic receptors 5HT1a and 5HT2a, opioid and dopamine receptors, ionotropic receptors including transient receptor potential (TRP) channels, GABA receptors, ion channels, transporters and enzymes including COX1 and 2 (Mlost, Bryk, and Starowicz 2020), all of which have implicated in pain processing.

### CBD and Inflammation

Nonetheless, evidence for CBD as a standalone analgesic remains to be sparse. Its role as an anti-inflammatory drug is much better substantiated. First, in preclinical mouse models of arthritis, oral CBD reduced joint swelling, prevented severe histopathological joint damage, and reduced pro-inflammatory cytokines, including TNF (Malfait et al. 2000). Additional studies by multiple groups have since established that CBD has a broad ranging effect on immune responses, most likely via signalling through the endocannabinoid system, adenosine A_{2A}, PPAR gamma, 5-HT1a and GPR55 receptors (Burstein 2015; Nichols and Kaplan 2020). Clinical studies indicated that CBD may have clinical potential in the treatment of many autoimmune diseases, apart from RA and MS, such as autoimmune hepatitis, experimental autoimmune myocarditis, and autoimmune diabetes.

The ability of CBD to modulate TNF and reactive oxidative stress offers the potential to implement it in treating a variety of complex inflammatory and metabolic conditions, including diabetes, diabetes-related cardiomyopathy, cardiovascular diseases (arrhythmia, atherosclerosis, hypertension and stroke), and cancer, arthritis, anxiety, psychosis, epilepsy, neurodegenerative disease (Alzheimer's) and a variety of skin diseases (Atalay, Jarocka-Karpowicz, and Skrzydlewska 2019).

### CBD and Anxiety

Following the observation in rodents that CBD can attenuate or even prevent the anxiogenic effects of high dose THC, many groups set out to evaluate the effects of CBD on anxiety induced behaviours (Crippa et al. 2018). The first of these demonstrated a significant anti-anxiety effect of low dose CBD in rats (2.5-10 mg/kg) using the elevated plus-maze model (Guimaraes et al. 1990). Notably the effects were lost at higher doses (20 mg/kg). This bell-shaped dose effect was later reproduced in human trials (Linares et al. 2019). Subsequently, several studies in rodents that modelled generalised anxiety, fear and panic disorder supported these initial findings. The anxiolytic effect of CBD in humans was first studied in healthy volunteers using the Simulated Public Speaking Test (SPST), where CBD (300 mg) was compared to two anxiolytic compounds, ipsapirone (5 mg) and diazepam (10 mg) in a double-blind, placebo-controlled trial. This study demonstrated that all compounds attenuated SPST anxiety, but unlike the others, CBD did not impact systolic blood pressure or sedation (Zuardi et al. 1993). Another study in patients with social anxiety disorder (SAD) demonstrated that patients treated with CBD (600 mg) had lower anxiety levels and lower negative self-assessment scores and fewer somatic symptoms compared to placebo (Bergamaschi et al. 2011). These finding were further supported by brain imaging studies (SPECT and fMRI), showing that the modulatory effects of CBD in limbic and paralimbic areas are compatible with the effects of known anxiolytic compounds (Batalla et al. 2014; Crippa et al. 2004; Fusar-Poli et al. 2009).

Consideration all these data highlights the need for rigorous clinical testing of CBD, especially in the triad of inflammation, pain and anxiety. There is no single molecular pathway that has been attributed to CBD, therefore, it is rather a combination of pathways that is responsible for the manifestation of its anti-inflammatory, analgesic and anxiolytic effects. Pain related conditions that may treated with CBD and/or THC are listed in Annex A.

Another important cannabinoid that is applicable for the treatment of pain and anxiety is THC, especially in combination with CBD or other cannabinoids or other drugs. The physicochemical properties of THC, CBD and other cannabinoids are similar in being highly lipophilic. Therefore, it is most likely that finding a successful formulation of CBD can be translated to THC and other cannabinoids and cannabinoid mixtures, with minor modifications accounting for particle size.

Another lipophilic drug of interest is Cyclosporin (Neoral, Deximune, Sandimmune, Gengraf), which in currently provided in a liquid formulation loaded in soft gelatine capsule. It is often taken to prevent rejection after organ transplantation or for inflammatory diseases.

Yet another drug of interest is Amphotericin B used for treating systemic fungal infections. It is also highly lipophilic and is delivered as a dispersion containing Cremophore, a PEG-hydrogenated castor oil that can have significant side effects.

### EXAMPLES

The following examples demonstrate some important features of the present pro-nano formulations (LPNFs and SPNFs) and their adaptability to various types of solid dosage forms.

### Example 1: LPNF-CBD Adsorbed into Porous Silica Particles

Typical liquid pro-nano formulation (LPNF) is composed of liquid lipids (e.g., mono-, di- and triglycerides, fatty alcohol), liquid surfactants or/and emulsifiers (e.g., Tween, Span and others), PEG conjugates with fatty acids and solvents. The active (10% CBD solution w/w) is dissolved in the LPNF blank formulation.

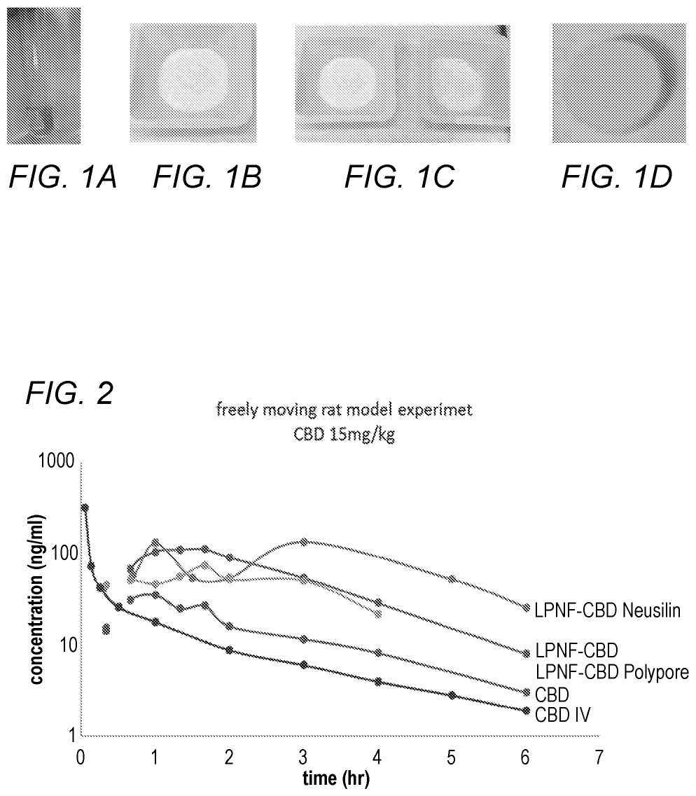

In a pilot study, LPNF-CBD was adsorbed into silica powder (Neusilin) at 0.5 ratio w/v. Adsorbed powder was mixed with hydroxypropyl methylcellulose (HPMC) and tableting aid (Syloid® 244 FP). Tablets were prepared by direct compression using a 1.3 cm die. FIG. 1 illustrates the main steps (1a-1d) in the preparation process.

Subsequent studies of the CBD release in the simulated gastric and intestinal fluids indicated constant release of <100 nm nanoparticles with CBD for at least 17 h (see below). Pharmacokinetic study in rats receiving oral administration of the LPNF-CBD powder dispersed in water showed significant CBD blood levels (see below).

### Example 2: CBD Release from LPNF Adsorbed into Porous Silica Particles

The CBD release from the LPNF-CBD tablet was tested in the USP simulated gastric fluid (pH=1.2) for 2 h and in the USP simulated intestinal fluid (pH=6.8) with 0.5% sodium lauryl sulphate (SLS) w/w for 48 h, thus mimicking the conditions in the GI tract.

### Materials and Methods

For USP simulated gastric fluid, 3 gr sodium chloride (NaCl) were dissolved in 10.5 mL of hydrochloric acid (HCl) and distilled water up to 1500 mL, pH was adjusted to pH=1.2.

For USP simulated intestinal fluid, 0.5% SLS w/w were prepared from a buffer solution with 10.34 gr sodium phosphate monobasic monohydrate in 500 mL water, 23.1 mL IN sodium hydroxide and distilled water up to 1500 mL, pH was adjusted to pH=6.8, upon which 7.5 gr SLS was added to the solution.

For LPNF-CBD preparation, CBD (10% w/w) was added to the LPNF-blank (90% w/w) and mixed for few min at 37° C. until complete dissolution. The non-active ingredients in the LPNF-blank are listed in Table 1.

TABLE 1
Non-active ingredients in the LPNF-blank

| Excipient | Percentage w/w |
|---|---|
| Tween 20 | 14.1 |
| Span 80 | 14.1 |
| Lecithin | 8.3 |
| Tricarpin | 14.1 |
| Hydrogeneated castor oil (HCO 40) | 14.1 |
| Ethyl lactate | 35.4 |
| SUM | 100.0 |

For tablets, LPNF-CBD was blended with the silica powder (Neusilin US2) using a mortar and a pestle to obtain a homogenous dry powder. HPMC and a tableting aid (Syloid 244 FP) were added and mixed. To produce tablets, 0.55 gr mixture was fed into the die of an instrumented single punch tableting machine (Perkin Elmer) with a 1.3 cm flat faced punch. Tablets were prepared by direct compressing under the compression force of 3 ton for 1 min. Tablets were weighed after compressing and kept in 4° C. The tablets included non-active ingredients as in Table 2 and 25 mg of CBD. Additional tablets were prepared with no HPMC and Syloid, or higher concentrations of HPMC and Syloid for CBD release studies.

TABLE 2
Non-active ingredients in the tablets made of adsorbed LPNF

| | Percentage | Weight (mg) | Weight (mg) |
|---|---|---|---|
| Material | w/w | per 1 tablet | per 5 tablets |
| LPNF | 45.5 | 250 | 1250 |
| Neusilin US2 | 22.7 | 125 | 625 |
| HPMC | 30.0 | 165 | 825 |
| Syloid 244 FP | 1.8 | 10 | 50 |
| SUM | 100.0 | 550 | 2750 |

The CBD dissolution test was performed using ERWEKA DT 6 R in 250 mL buffer at 37° C. and 50 rpm. Tablets in the experimental group (N=3) were incubated in simulated gastric fluid for 2 h and in simulated intestinal fluid for 48 h. Particle size and CBD release were analysed at specific time points (every hour for 12 h and at 24 h) by Dynamic Light Scattering (DLS) and HPLC, respectively, using established protocols. At the end of the experiment (48 h), remnants of the tablets were extracted using ethyl acetate.

### Results and Conclusions

The percentage of CBD released from the tablets into the simulation solution was calculated relative to the CBD concentration of 100 ug/mL as 100% (25 mg CBD in 250 mL simulation solution). The study included 30% HPMC as in Table 3, and 0, 5%, 10%, 20% HPMC tablets. In all tested tablets, CBD release was constant. The release rate was correlated with the HPMC content, with tablets with lower HPMC reaching CBD peaks faster than the 30% HPMC tablets up to the maximum of 60% CBD release. The release rate at pH=2 was similar to pH=6.8. The particle size was maintained below 50 nm, irrespective of pH and HPMC content.

This study suggested that LPNF alone or together with lipophilic active(s) can be absorbed into porous silica particles to produce successful solid dosage forms, and that the HPMC content of the dosage form can be modulated to provide controlled release of the nanoparticles with the lipophilic active(s) into aqueous solution mimicking the conditions in the GI tract.

### Example 3: Loading LPNF into Inert Solid Porous Agents

Several inert solid porous agents were tested for their capacity to adsorb LPNF. Selected candidates were used to produce the powder forms of LPNF-CBD and were further tested in pharmacokinetic studies.

### Materials and Methods

Three porous agents were tested:
1. Neusilin US2 is a synthetic amorphic derivative of magnesium aluminometasilicate, having an average particle size of 106 μm and oil adsorbing capacity of 2.7-3.4 ml/gr. It has the advantage in making powders with better flow and compressibility properties compared to smaller silica particles such as Aerosil 200. Larger particles have greater mechanical force during tablet compression.
2. Silica gel 60 is the commonly used silica with particles size in the range of 60-200 μm.
3. Polypore E200, allyl methacrylate crosspolymer, is a multi-functional adsorbent polymer.

For LPNF-blank, a stock preparation of LPNF-blank was prepared from the ingredients in Table 1 to form a clear uniform solution. The solution was stable at 0 to 37° C. for months.

For determining LPNF load in adsorbent, selected adsorbents were weighed and added in measured increments to 1 mL LPNF-blank until totally adsorbed and flowing powder was formed.

For nanoparticles size and distribution, selected LPNF powders were added to 9 mL pre-heated water (37° C.) and mixed for 2 min. The supernatant was filtered and analyzed by a Zetasizer using a previously established protocol.

For pharmacokinetic analysis of LPNF-CBD powders, male Wistar rats were tested in three groups: a control group received LPNF-CBD prepared as in EXAMPLE 2 (N=5) with CBD concentration 2 mg/mL, administered by oral gavage; experimental groups received LPNF-CBD Polypore or Neusilin US2 powders dispersed in water (N=4 and N=5, respectively) with LPNF-CBD concentration of 20 mg/ml, administered by oral gavage; control groups received CBD in propylene glycol:ethanol:water (4.5:4.5:1 v/v) with CBD concentration of 3 mg/mL, administered by oral gavage or intravenously (N=5 and N=3, respectively). In all groups, the administered CBD dose was 15 mg/kg. For pharmacokinetic (PK) studies, blood samples (0.35 mL) were taken at 0, 0.3 h, 0.6 h, 1 h, 1.5 h, 2 h, 3 h, 4 h and 6 h time points. Plasma was separated by centrifugation and stored frozen until LCMS analysis.

### Results and Conclusions

The maximum LPNF load and adsorption into selected absorbers/carriers are shown in Table 3. Polypore showed the best absorption capacity, followed by Neusilin US2.

TABLE 3
LPNF-blank load and adsorption into the solid adsorbents

| Carrier | LPNF load mg/gr |
|---|---|
| Neusilin US2 | 2 |
| Polypore | 3.63 |
| Silica gel | 1.67 |

The analysis of particles' size released upon dispersion of the LPNF Polypore and Neusilin powders in water is shown in Table 4, suggesting that both absorbents released nanoparticles of <100 nm and mostly <50 nm.

TABLE 4
Average particle size as function of time contact with water
Particle size (nm)

| Time | Polypore | Neusilin US2 |
|---|---|---|
| 0.17 | 26.49 | 77.14 |
| 0.50 | 25.92 | 79.31 |
| 1.00 | 26.85 | 65.87 |
| 2.00 | 26.61 | 62.83 |
| 4.00 | 28.87 | 76.17 |
| 8.00 | 31.43 | 46.35 |
| AVR | 27.69 | 67.9 |
| SD | 2.09 | 12.5 |

PK study was conducted in the freely moving rat model (in-vivo) in five groups of animals receiving: (1) CBD in propylene glycol:ethanol:water solution; (2) LPNF-CBD, (3) LPNF-CBD Polypore and (4) LPNF-CBD Neusilin powders dispersed in water; all administered by oral gavage; and (5) CBD in propylene glycol:ethanol:water solution administered IV. The respective PK profiles are shown in FIG. 2 (log scale), the resulting PK parameters are summarized in Table 5.

TABLE 5
PK parameters in the five tested groups

| | | | | Liner | |
|---|---|---|---|---|---|
| | | | | terminal | Elimination |
| | AUC | Cmax | Cmax_2 | slope | half- |
| | (ng*h/mL) | (ng/mL) | (ng/mL) | (h⁻¹) | life (h) |
| CBD | 158 ± | 39 ± | | 0.2 | 3.2 |
| | 24 | 8 | | | |
| LPNF-CBD | 251 ± | 137 ± | | 0.3 | 2.5 |
| | 77 | 43 | | | |
| LPNF-CBD | 135 ± | 51 ± | 74 ± | 0.4 | 1.9 |
| Polypore | 53 | 10 | 42 | | |
| LPNF-CBD | 397 ± | 131 ± | 135 ± | 0.3 | 2.2 |
| Neusilin US2 | 55 | 25 | 40 | | |
| CBD IV | | | | 0.2 | 4.1 |

(marked) significant difference (p < 0.05) compared to CBD

Comparing the groups receiving oral preparations, the LPNF-CBD Neusilin solid formulation had the highest AUC value, suggesting a higher drug exposure and overall better PK performance of this preparation. In this experiment, the AUC of LPNF-CBD Neusilin was 1.5-fold higher than the AUC of the CBD in solution (statistically significant) and was higher than the AUC of the liquid dosage form LPNF-CBD (not reaching statistical significance). Although it does not prove a better PK performance that the liquid formulation, it still indicative as to the lack of interference of the added adsorption and solidification processes with the original properties of LPNF.

Comparing the liquid and the solid LPNF, the PK profiles of the solid forms were characterized by two peaks compared to single peak profiles in the liquid form. There were also differences in the CBD absorption profiles, which decreased faster in the liquid form (after 1.6 h) and slower in the solid forms (after 3 h). The liner terminal slope and the elimination time were similar, irrespective of formulations and administration routes.

Taken together, these studies show that LPNF can be successfully adsorbed into silica or polymer particles to produce solid formulations that retain the ability to form nanoparticles in aqueous media, and as a result, all the ensuing benefits of improved solubility, PK performance and oral bioavailability. Of the tested adsorbents, Neusilin US2 and Polypore had the best oil/powder ratios. As these two represent two different types of materials, porous silica and polymeric scaffold, it suggests potential use of a wide range of adsorbents. In addition, the current studies in-vivo have provided preliminary evidence that the produced solid formulations can delay CBD absorption, in other words, they have the potential to provide controlled or sustained release dosage forms.

### Example 4: Adsorbed LPNF with Other Non-Active Ingredients and CBD

This study explored different types of surfactants, lipids and adsorbents to produce more effective and stable adsorbed LPNF formulations.

### Materials and Methods

LPNF used Koliphore HS 15 (polyethylene glycol mono- and di-esters of 12-hydroxystearic acid (lipophilic part) and about 30% of polyethylene glycol (hydrophilic part), polyoxyl 15 hydrostearate (Solutol) and Koliphore RH 40 (polyethylene glycol conjugated to hydrogenated castor oil) obtained from BASF Pharma; sesame oil from Across Organics; Tween 80 (polyoxyethylene sorbitan monolaurate), Span 80 (sorbitan monooleate), and propylene glycol from Sigma Aldrich; and synthetic CBD powder from PureForm Global.

Powder absorbents used Syloid 244 FP (mesoporous silicas) from GRACE; Neusilin US2 (synthetic magnesium aluminometasilicate) from Fuji Chemical Industries; Microcrystalline cellulose (MCC) (Avicel), ethyl cellulose (EC) from Carl Roth; mannitol, Carbopol 971 NF from Lubrizol; Eudragit S100 from Evonik; Charcoal from Bio Lab; and corn starch from CS chemicals.

LPNF preparation was made by mixing sesame oil, Koliphore HS 15, Koliphore RH 40, propylene glycol, Span 80 and Tween 20 in predetermined weight ratios at 37° C.

LPNF-CBD preparation was made from 100 mg CBD powder dissolved into 1 ml LPNF (10% CBD w/w). LPNF density is approximately 1 g/ml.

For powder absorption, different powder absorbents were slowly added to 1 ml LPNF or LPNF-CBD (N=10) up to the ratio 1:1 or until fully absorbed without feeling oily to touch.

For tablet preparation, tablets were prepared by compressing 750 mg powder using 13 mm tablet mold under 2 tons pressure.

For CBD release in simulated gastric solution, 200 mg powders and tablets were dispersed in 40 ml 0.1M HCl (pH˜2) at 37° C. and 75 rpm, after 2 h the medium was replaced with 40 ml PBS solution (PH˜7).

CBD content was determined by HPLC-UV using column Luna C-18(2), 5 μm, 150×4.6 mm, 100 Å Phenomenex, X00F-4252-E0 under the conditions of mobile phase of 5 mM NaH₂PO₄ in water pH 3.0/acetonitrile ratio of 20:80; column temperature of 35° C.±5° C.; UV monitoring wavelength of 211 nm; and CBD RT of 4.6 min. Standards preparation for HPLC: used 15 mg/ml CBD stock solution in methanol diluted to 0.5-100 μg/ml with 0.5% w/v SLS.

### Results and Conclusions

LPNF-blank was prepared from non-active ingredients listed in Table 6.

TABLE 6
Non-active ingredients in LPNF-blank

| Material | Weight [g] | Percentage |
|---|---|---|
| Sesame oil | 2.0 | 10% |
| Koliphore HS 15 | 1.6 | 8% |
| Koliphor RH 40 | 4.0 | 20% |
| Propylene Glycol | 4.2 | 21% |
| Span 80 | 4.1 | 21% |
| Tween 20 | 4.1 | 21% |
| Total weight [g] | 20.0 | 100% |

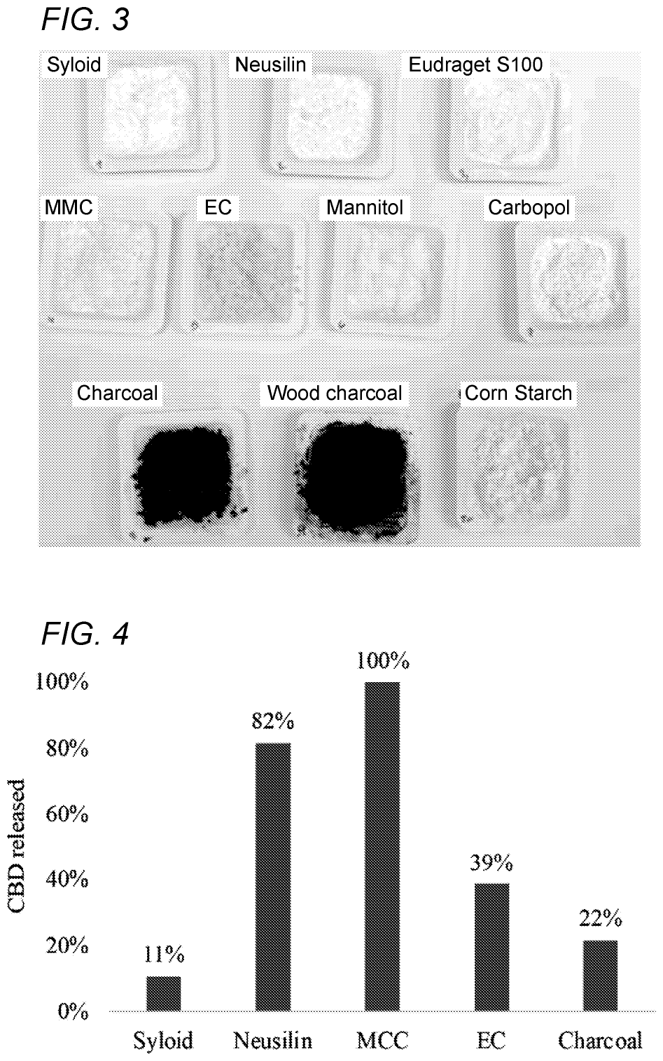

The different powder absorbents that were added to LPNF-blank and tested for adsorpsion capacity are listed Table 7. The absorbents with the best results in terms of adsorpsion capacity, dryness and texture were Syloid, Neusilin, MCC, EC and Charcoal. Illustration of the results is provided in FIG. 3.

TABLE 7
Powders produced with different absorbents and LPNF-blank

| | Material | Ratio w/v | Appearance |
|---|---|---|---|
| 1 | Syloid | 0.5 | dry powder |
| 2 | Neusilin | 0.5 | dry powder |
| 3 | Eudragit (acrylic copolymer) | 1.0 | dough like |
| 4 | Microcrystalline cellulose (MCC) | 1.0 | dry powder |
| 5 | Ethyl cellulose (EC) | 1.0 | dry powder |
| 6 | Mannitol | 1.0 | Paste |
| 7 | Carbopol | 0.7 | dough like |
| 8 | Charcoal | 1.0 | dry powder |
| 9 | Wood Charcoal | 1.0 | Paste |
| 10 | Starch | 1.0 | Paste |

The absorbents with the best absorption capacity were used to produce respective LPNF-CBD powders, which were further tested for CBD loading, particle size and CBD release at neutral pH (200 mg powders in 40 ml PBS pH=7). The analyses of CBD loading, particles size and particle polydispersity index (PDI) are shown in Table 8 (after 24 h), and CBD release in FIG. 4 (after 2 h).

TABLE 8
CBD loading, particles size and PDI of the LPNF-CBD powders

| | CBD | Particles size | | Particles PDI |
|---|---|---|---|---|
| Material | content (w/w) | of after 24 h | | after 24 h |
| Syloid | 8% | 209 ± 45 | nm | 0.46 |
| Neusilin | 8% | 56 ± 13 | nm | 0.22 |
| MCC | 5% | 39 ± 12 | nm | 0.23 |
| EC | 5% | 75 ± 18 | nm | 0.27 |
| Charcoal | 5% | 162 ± 43 | nm | 0.39 |

In terms of particles size, the best results were obtained with LPNF-CBD adsorbed into Neusilin and MCC. These two powders further showed almost complete CBD release, up to 82% and 100% after 2 h respectively, while the other powders showed the same release only after 24 h (data not shown). These finding served as a basis for producing more advanced adsorbed LPNF, with Neusilin, MCC and additional ingredients.

Tablets were produced from LPNF adsorbed into Neusilin and MCC as above and additional ingredients listed in Table 9, specifically lactose and/or HPMC.

TABLE 9
Various compositions of the tablets made of adsorbed LPNF

| | PNL powder | Lactose | Neusilin | MCC | HPMC | Total | CBD loading |
|---|---|---|---|---|---|---|---|
| Sample code | [mg] | [mg] | [mg] | [mg] | [mg] | [mg] | [w/w] |
| MAE-3-19-A | 300 | — | — | 450 | — | 750 | 2.0% |
| MAE-3-19-B | 300 | — | 450 | — | — | 750 | 3.0% |
| MAE-3-24-A | 300 | 250 | 200 | — | — | 750 | 2.0% |
| MAE-3-24-B | 300 | 250 | — | 200 | — | 750 | 2.0% |
| MAE-3-27-A | 300 | 250 | 100 | — | 100 | 750 | 2.0% |
| MAE-3-27-B | 400 | 250 | — | 100 | — | 750 | 2.7% |
| MAE-3-27-C | 350 | 250 | — | 150 | — | 750 | 4.8% |
| MAE-3-27-D | 400 | 200 | 100 | — | 50 | 750 | 5.5% |
| MAE-3-27-E | 600 | — | — | — | 150 | 750 | 8.0% |
| MAE-3-27-F | 600 | 150 | — | — | — | 750 | 8.0% |

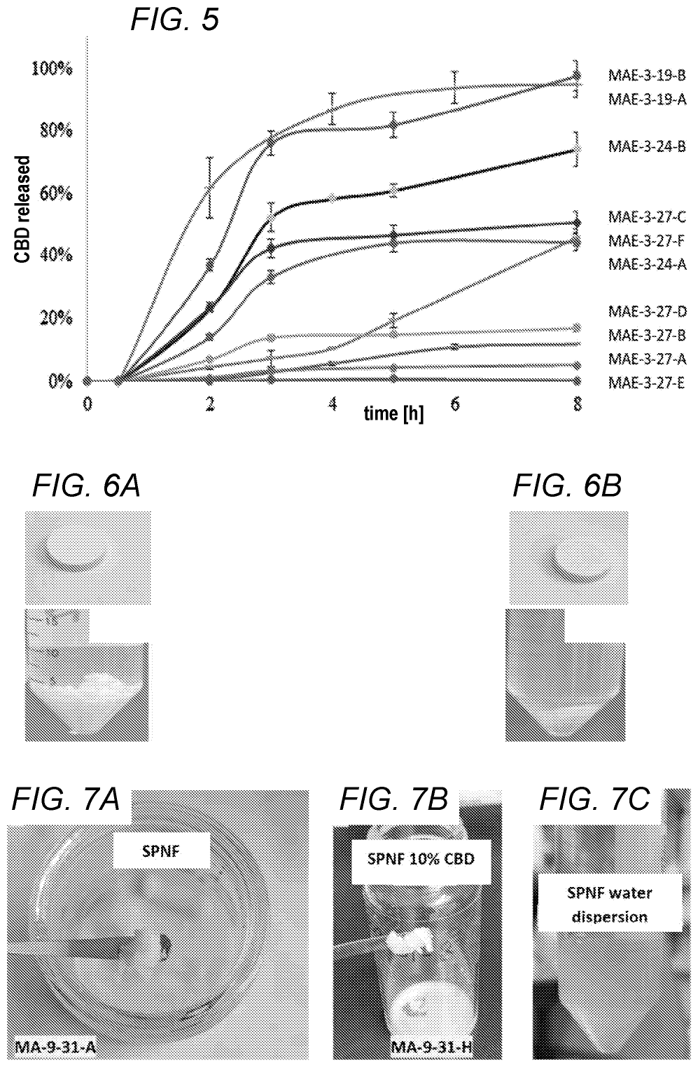

The tablets were further characterized for particle size and CBD release in the conditions mimicking the passage of the tablet in the GI tract, i.e., in simulated gastric solution (pH˜2) for 2 h and then in simulated intestinal solution (pH˜7) for additional 6 h (at several time points from 2 h to 8 h). The results are shown in Table 10 and FIG. 5.

TABLE 10
Tablets' particle size, PDI and
CBD release (at 2 h and 8 h time points)

| | Time | | | | | % CBD |
|---|---|---|---|---|---|---|
| Sample code | [h] | pH | size [nm] | | PDI | Released |
| MAE-3-19-A | 2 | 2 | 59 ± 2 | nm | 0.188 | 62% |
| | 8 | 7 | 55 ± 4 | nm | 0.234 | 94% |
| MAE-3-19-B | 2 | 2 | 98 ± 19 | nm | 0.677 | 0% |
| | 8 | 7 | 165 ± 10 | nm | 0.212 | 11% |
| MAE-3-24-A | 2 | 2 | 155 ± 11 | nm | 0.106 | 5% |
| | 8 | 7 | 196 ± 18 | nm | 0.289 | 50% |
| MAE-3-24-B | 2 | | 82 ± 9 | nm | 0.241 | 23% |
| | 8 | 7 | 95 ± 16 | nm | 0.304 | 75% |
| MAE-3-27-A | 2 | 2 | 171 ± 9 | nm | 0.47 | 1% |
| | 8 | 7 | 98 ± 7 | nm | 0.411 | 5% |
| MAE-3-27-B | 2 | 2 | 123 ± 6 | nm | 0.451 | 37% |
| | 8 | 7 | 100 ± 5 | nm | 0.362 | 98% |
| MAE-3-27-C | 2 | 2 | 222 ± 21 | nm | 0.649 | 23% |
| | 8 | 7 | 323 ± 22 | nm | 0.476 | 51% |
| MAE-3-27-D | 2 | 2 | 347 ± 41 | nm | 0.385 | 7% |
| | 8 | 7 | 275 ± 8 | nm | 0.46 | 17% |
| MAE-3-27-E | 2 | 2 | 867 ± 171 | nm | 0.722 | 0% |
| | 8 | 7 | 213 ± 7 | nm | 0.536 | 2% |
| MAE-3-27-F | 2 | 2 | 178 ± 58 | nm | 0.685 | 14% |
| | 8 | | 109 ± 12 | nm | 0.426 | 45% |

The results showed several interesting findings: (Tables 9-10 marked): (1) MAE-3-19-A with MCC as the only additive yielded the fastest CBD release reaching about 60% of the loaded CBD at the 2 h time point and about 95% at the 8 h time point, and particle size maintained at an average of 55-59 nm; (2) MAE-3-24-B with MCC and lactose yielded up to 75% release at the 8 h time point and particles of 82-95 nm; (3) MAE-3-27-B with the same lactose but a lower MCC yielded up to 98% CBD release at the 8 h time point but larger particles of 100-120 nm; (4) MAE-3-27-F a lower lactose and no MCC yielded only up to 45% CBD at the 8 h time point with larger particles of 110-180 nm. The HPMC tablets yielded significantly lower CBD release than the MCC tablets, most likely due to lower disintegration. The Neusilin tablets were generally more unstable in terms of particle size and had lower CBD release. FIG. 6 illustrates the appearance of Neusilin and MCC tablets, MAE-3-19-A and MAE-3-19-B, respectively, at 0 and the 6 h time point.

Altogether the results prove the feasibility of making solid LPNF forms, using powder adsorbents such as MCC and additional non-active ingredients to provide efficient release of lipophilic actives for at least 8 h after administration, while maintaining a nanometric particles size at the range of less than 100 nm.

### Example 5: SPNF with CBD and Other Lipophilic Actives

This study explored the possibly of using solid in active ingredients, and specifically surfactants, emulsifiers and lipids, to produce more advanced and stable solid pro-nano formulations (SPNF).

### Materials and Methods

SPNF preparations used IMWITOR® 900 K (solid wax); COMPRITOL 888 ATO; Witepsol e 85; Precirol ATO 5; Novata wax; Softisan 378; Tween 80; Kollipor RH 40; Kollipor HS 15; PEG 1000; PEG 400; Labrafil® M 1944; Lutrol F 68; Lecithin; Miglyol (MCT); Labrasol®; Labrafil® M 1944; Transcutol; Poly(vinylpolypyrrolidone) cross-linked; lactose; mannitol, CBD synthetic powder, Curcumin powder, Ibuprofen, Oxybenzone, 7-Nitroinidazole (7-NI).

IMWITOR® 900 K is mostly composed of monoesters of stearic acid and palmitic acid with glycerol, with a melting point about 61° C. It does not contain additives or residues of antioxidants, stabilizers or solvents. It exhibits high storage stability and no risk of rancidity because it is free from unsaturated components. It is classified as GRAS by the FDA, and therefore, is widely used in pharmaceutical oral solid dosage forms as lipophilic matrix, tablet lubricant, emulsion stabilizer, dispersing agent for pigments and for sustained release.

IMWITOR® 372 P is a partially neutralized ester of plant derived monoglycerides and diglycerides, saturated edible fatty acids with citric acid. It is free from polyethylene glycols and its monomers and contains no residues of solvents or stabilizers. It is partly ionic, oil soluble emulsifier, similar to lecithin. It is food approved in Europe.

Labrafil® M 1944 is oleoyl polyoxyl-6 glycerides, a nonionic water-dispersible surfactant that self-emulsifies in aqueous media to form a coarse emulsion (SEDDS). It is widely used in lipid-based formulations to solubilize and increase oral bioavailability of poorly water-soluble actives and as a co-emulsifier in topical formulations to improve stability.

Labrasol® is a PEG derivative of medium chain fatty acid triglyceride of capric and caprylic acid (caprylocaproyl polyoxyl-8 glycerides). It consists of a small fraction of mono-, di- and triglycerides and mainly PEG-8 (MW 400) mono- and diesters of caprylic (C8) and capric (C10) acids. It is widely used in SNEDDS (nano) formulations due to its good solubilizing power and spontaneous self-emulsification ability.

For SPNF-blank preparation, the respective ingredients were weighed in a glass vial and heated at 100° C. for 20 min while stirring until complete melting, dissolution and/or homogeneity. The mixture was cooled to room temperature for 1 h.

For SPNF-drug preparation, 10% w/w drug was added to the SPNL-blank preparation and stirred until complete dissolution of the drug.

### Results and Conclusions

Several types SPNF-drug formulations were prepared using different combinations of active and non-active ingredients, and for tested for appearance, texture and particle size at 37° C. The results are shown in Table 11.

TABLE 11
Composition and characteristics of various SPNF-drug formulations

| | | | Particle size |
|---|---|---|---|
| Sample code/ | Transparency in | | in water |
| composition | molten state | Lipophilic drug | at 37° C. |
| MA-9-3-D | Transparent liquid | Curcumin, CBD | <200 nm |
| Imwitor 900 k - 50% | at >65° C. | | |
| Tween 80 - 10% | White waxy solid | | |
| Kolliphor RH 40 - 10% | MP >60° C. | | |
| PEG 400 - 10% | | | |
| Kolliphor HS 15 - 10% | | | |
| PEG 1000 - 10% | | | |
| MA-9-7-A | Transparent liquid | Paclitaxel | <200 nm |
| Imwitor 900 k - 50% | at >65° C. | | |
| Tween 80 - 10% | White waxy solid | | |
| Kolliphor RH 40 -10% | MP >60° C. | | |
| PEG 400 - 10% | | | |
| Castor oil - 20% | | | |
| MA-9-7-B | Transparent liquid | CBD | <200 nm |
| Imwitor 900 k - 40% | at >65° C. | | |
| Tween 80 - 10% | White waxy solid | | |
| Kolliphor RH 40 -40% | MP >60° C. | | |
| PEG 400 - 10% | | | |
| MA-9-7-C | Transparent liquid | CBD, curcumin | 220 ± 19.02 nm |
| Imwitor 900 k - 40% | at >65° C. | | PDI: 0.330 ± 0.053 |
| Tween 80 - 10% | White waxy solid | | |
| Kolliphor RH 40 -10% | MP >60° C. | | |
| PEG 1000 - 10% | | | |
| Castor oil - 30% | | | |
| MA-9-10-A | Transparent liquid | CBD, paclitaxel | 662.8 ± 220.8 nm |
| Imwitor 900 k - 40% | at >65° C. | | PDI: 0.606 ± 0.344 |
| Tween 80 - 10% | White waxy solid | | |
| Kolliphor RH 40 -10% | MP >60° C. | | |
| PEG 1000 - 10% | | | |
| Castor oil - 30% | | | |
| MA-9-10-B | Transparent liquid | Curcumin | 959.5 ± 356.9 nm |
| Imwitor 900 k - 40% | at >65° C. | | PDI: 0.870 ± 0.159 |
| Tween 80 - 10% | White waxy solid | | |
| Kolliphor RH 40 -10% | MP >60° C. | | |
| PEG 1000 - 10% | | | |
| Castor oil - 30% | | | |
| MA-9-13-A | Transparent liquid | Various lipophilic | 415.4 ± 27.36 nm |
| Imwitor 900 k - 40% | at >65° C. | agents | PDI: 0.809 ± 0.294 |
| Tween 80 - 10% | White waxy solid | | |
| Kolliphor RH 40 -10% | MP >60° C. | | |
| PEG 1000 - 10% | | | |
| Castor oil - 20% | | | |
| Novata wax - 10% | | | |
| MA-9-13-B | Transparent liquid | Various lipophilic | CBD: 285.4 ± 21.93 nm |
| Imwitor 900 k - 40% | at >65° C. | agents | PDI: 0.124 ± 0.142 |
| Tween 80 - 10% | White waxy solid | | |
| Kolliphor RH 40 -10% | MP >60° C. | | |
| PEG 1000 - 10% | | | |
| Castor oil - 20% | | | |
| Softisan 378 - 10% | | | |
| MA-9-13-C | Transparent liquid | Various lipophilic | Disperse well |
| Imwitor 900 k - 40% | at >65° C. | agents | 430.8 ± 80.03 nm |
| Tween 80 - 10% | White waxy solid | | PDI: 0.784 ± 0.305 |
| Kolliphor RH 40 -10% | MP >60° C. | | |
| PEG 1000 - 10% | | | |
| Castor oil - 20% | | | |
| Lutrol F 68 - 10% | | | |
| MA-9-13-D | Transparent liquid | Various lipophilic | <200 nm |
| Imwitor 900 k - 40% | at >65° C. | agents | |
| Tween 80 - 20% | White waxy solid | | |
| Kolliphor RH 40 -10% | MP >60° C. | | |
| PEG 1000 - 10% | | | |
| Castor oil - 20% | | | |
| MA-9-19-A | Transparent liquid | Various lipophilic | Disperse well with |
| Imwitor 900 k - 40% | at >65° C. | agents | traces of aggregates |
| Tween 80 - 10% | White waxy solid | | |
| Kolliphor RH 40 -10% | MP >60° C. | | |
| PEG 1000 - 10% | | | |
| Castor oil - 20% | | | |
| Oleic acid - 10% | | | |
| A-9-19-B | Transparent liquid | Various lipophilic | Disperse well with |
| Imwitor 900 k - 40% | at >65° C. | agents | traces of aggregates |
| Tween 80 - 10% | White waxy solid | | |
| Kolliphor RH 40 -10% | MP >60° C. | | |
| PEG 1000 - 10% | | | |
| Castor oil - 20% | | | |
| Labrasol - 10% | | | |
| MA-9-19-C | Transparent liquid | Various lipophilic | Disperse well with |
| Imwitor 900 k - 40% | at >65° C. | agents | traces of aggregates |
| Tween 80 - 10% | White waxy solid | | |
| Kolliphor RH 40 -10% | MP >60° C. | | |
| PEG 1000 - 10% | | | |
| Castor oil - 20% | | | |
| Transcutol - 10% | | | |
| MA-9-19-F | Transparent liquid | CBD or Curcumin | Disperse well |
| Imwitor 900 k - 40% | at >65° C. | | Size: 177.8 ± 5.122 nm |
| Tween 80 - 10% | White waxy solid | | PDI: 0.244 ± 0.034 |
| Kolliphor RH 40 -10% | MP >60° C. | | |
| PEG 1000 - 10% | | | |
| Labrafil M 1944 - 30% | | | |
| MA-9-22-A | Transparent liquid | CBD | Disperse well |
| Imwitor 900 k - 40% | at >65° C. | | Size: 110.2 ± 0.451 nm |
| Tween 80 - 10% | White waxy solid | | PDI: 0.237 ± 0.017 |
| Kolliphor RH 40 -10% | MP >60° C. | | |
| PEG 1000 - 10% | | | |
| Labrafil M 1944 - 30% | | | |
| MA-9-22-B | Transparent liquid | Curcumin | Disperse well |
| Imwitor 900 k - 40% | at >65° C. | | Size: 133.5 ± 15.61 nm |
| Tween 80 - 10% | White waxy solid | | PDI: 0.399 ± 0.065 |
| Kolliphor RH 40 - 10% | MP >60° C. | | |
| PEG 1000 - 10% | | | |
| Labrafil M 1944 - 30% | | | |

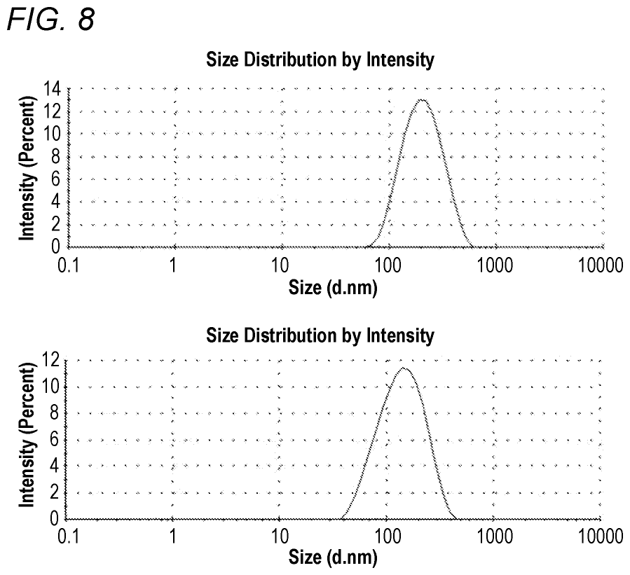

A prototype SPNF (MA-9-31-A) was constructed basing on the formulations with the optimal results (Table 11 marked). The non-active ingredients in the prototype SPNF are listed in Table 12. The visual appearance of the respective SPNF-blank and SPNF-CBD and its water dispersion of the SPNF-blank are shown in FIG. 7, the respective particle size profiles are shown in FIG. 8.

TABLE 12
Composition of the non-active ingredients in MA-9-31-A

| Material | % w/w |
|---|---|
| IMWITOR ® 900 K | 40 |
| Tween 80 | 10 |
| Kollipor RH 40 | 10 |
| PEG 1000 | 10 |

In summary, the data suggests that the prototype SPNF preserves an attractive appearance and texture of solid white wax, as a blank formulation and also with the addition of the active ingredient. Both were easily dispersed in water at 37° C. Additional experiment at 45° C. overnight suggested that the appearance and texture were also preserved under these conditions (data not shown). Particle size analysis of the water dispersions of the SPNF-blank and SPNF-CBD by dynamic light scattering (DLS) yielded particle size of 177.8±5.1 nm and 139.7±3.7 nm, respectively. This latter was rather surprising.

The effect of the addition of a dispersing agent on particle size was further examined. The results are shown in Table 13. The best results were obtained with SPNF-CBD and 50% crosslinked povidone (particle size of about 116 nm). More generally, increased amounts of a dispersing agent correlated with reduced particle size.

TABLE 13
The effect of dispersing agents on particle size of SPNLs

| Sample code | Sample | Dispersing agent % w/w | Size (nm) | PDI |
|---|---|---|---|---|
| MA-9-19-F | SPNF | — | 177.8 ± 5.1 | 0.244 ± 0.034 |
| MA-9-31-B | SPNF | 10% crosslinked povidone | 215.6 ± 16.5 | 0.219 ± 0.089 |
| MA-9-31-C | SPNF | 50% crosslinked povidone | 143.5 ± 6.3 | 0.201 ± 0.041 |
| MA-9-31-D | SPNF | 10% mannitol | 189.6 ± 5.3 | 0.195 ± 0.116 |
| MA-9-31-E | SPNF | 50% mannitol | 148.3 ± 4.5 | 0.219 ± 0.014 |
| MA-9-31-F | SPNF | 10% lactose | 205.2 ± 17.18 | 0.381 ± 0.067 |
| MA-9-31-G | SPNF | 50% lactose | 173.5 ± 23.75 | 0.269 ± 0.018 |
| MA-9-31-H | SPNL-CBD | — | 139.7 ± 3.7 | 0.326 ± 0.054 |
| MA-9-31-I | SPNF-CBD | 50% crosslinked povidone | 116.7 ± 3.4 | 0.233 ± 0.017 |
| MA-9-31-J | SPNF-CBD | 50% mannitol | 134.8 ± 4.4 | 0.205 ± 0.030 |
| MA-9-31-K | SPNF-CBD | 50% lactose | 171.4 ± 7.6 | 0.368 ± 0.976 |

The SPNFs with various dispersing agents (100 mg) were further subjected to dissolution analysis in water at 40° C. The results are shown in Table 14. One of the surprising findings was that the plain SPNF-CBD without any dispersing agents had the fastest time to complete dissolution.

TABLE 14
Dissolution analysis of SPNFs with
dispersing agents in water at 40° C.

| | | Dispersing | Time to complete | |
|---|---|---|---|---|
| Sample code | Sample | agent % w/w | dissolution | |
| MA-9-31-A | SPNF | — | 15 | min |
| MA-9-31-B | SPNF | 10% crosslinked | 6 | min |
| | | povidone | | |
| MA-9-31-C | SPNF | 50% crosslinked | 20 | min |
| | | povidone | | |
| MA-9-31-D | SPNF | 10% mannitol | 15 | min |
| MA-9-31-E | SPNF | 50% mannitol | 13 | min |
| MA-9-31-F | SPNF | 10% lactose | 11 | min |
| MA-9-31-G | SPNF | 50% lactose | 16 | min |
| MA-9-31-H | SPNF-CBD | — | 5 | min |
| MA-9-31-I | SPNF-CBD | 50% crosslinked | 22 | min |
| | | povidone | | |
| MA-9-31-J | SPNF-CBD | 50% mannitol | 10 | min |
| MA-9-31-K | SPNF-CBD | 50% lactose | 5 | min |

The effect of various lipophilic drugs was also studied with preparations of SPNF-blank and SPNF-drug (10% CBD, Curcumin, Ibuprofen, Oxybenzone or 5% 7-Nitro indazole, 7-NI). The results are shown in Table 15. Apart from 7-NI, all other SPNF-drug forms dissolved faster than SPNF-blank. The SPNF-CBD and SPNF-Oxybenzone maintained particle size at about 140 nm.

TABLE 15
Particle size and dissolution analyses of SPNFs with various drugs

| | | | | Time to complete | |
|---|---|---|---|---|---|
| Sample code | Sample | Size | PDI | dissolution | |
| MA-9-31-A | SPNF | 177.8 ± 5.1 | 0.244 ± 0.034 | 15 | min |
| MA-9-31-H | SPNF-CBD | 139.7 ± 3.7 | 0.326 ± 0.054 | 5 | min |
| MA-9-31-L | SPNF-Curcumin | 243.1 ± 41.6 | 0.581 ± 0.156 | 5 | min |
| MA-9-48- | SPNF-Ibuprofen | 154.1 ± 6.1 | 0.332 ± 0.070 | 5 | min |
| MA-9-31-J | SPNF-Oxybenzone | 142.1 ± 1.4 | 0.243 ± 0.008 | 6 | min |
| MA-9-31-K | SPNF-7NI | 154.3 ± 2.6 | 0.242 ± 0.009 | 20 | min |

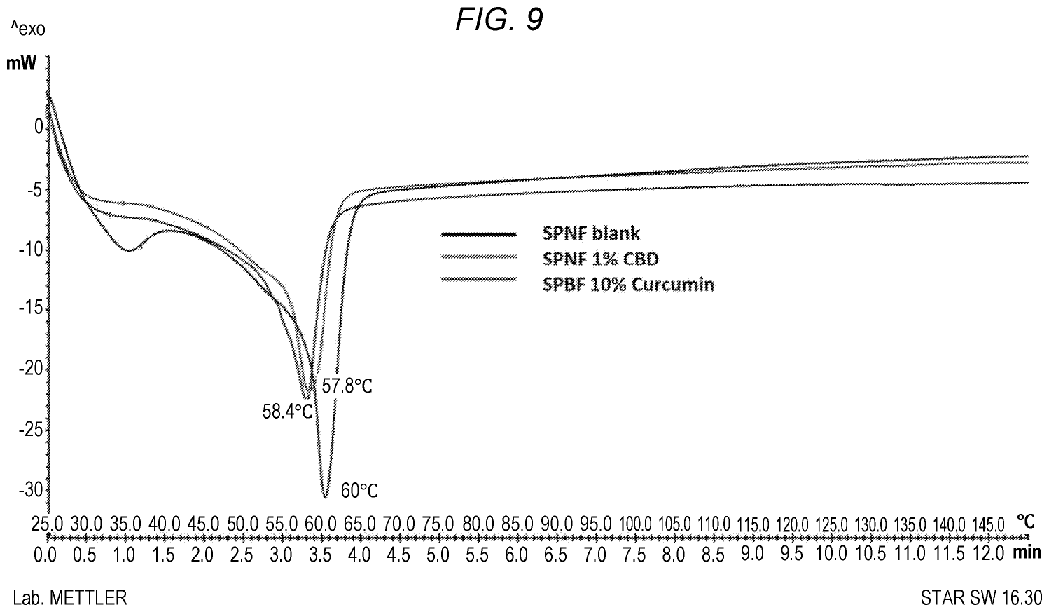

Homogeneity and texture of selected SPNFs (SPNF-blank, SPNF-CBD and SPNF-Curcumin) were further examined by melting point analysis using differential scanning calorimetry (DSC). The results are shown in FIG. 9. In all samples, the melting profiles exhibited a single endotherm at about 60° C., suggesting homogeneity with no crystallization and a complete dissolution of the drug.

In summary, this study demonstrates the feasibility of making solid nanoparticles releasing formulations (SPNF) from combinations of solid non-active components, i.e., lipids, surfactants, emulsifiers, adsorbents and other additives. The produced SPNF forms remained solid at room temperature, and upon dispersion in aqueous solution at 37° C. (mimicking the body temperature) released nanostructures with particle size of <200 nm. This is significant, since this specific property is responsible for a series of other advantages characteristic of SPNFs, such as improved stability, dissolution, PK performance and oral bioavailability. In addition, the produced SPNFs were compatible with several types of lipophilic actives, while retaining their particle size and other core properties.

### Example 6: Low Melting SPNF with Curcumin

Low melting or soft SPNF can have specific advantages and commercial interest. This study attempted to produce blank and active loaded formulations with a semisolid texture and the same self-emulsifying properties upon dispersion in aqueous medium at 37° C. The melting point of the formulations were 40° C. as determined by DSC.

### Materials and Methods

For soft SPNF-blank, stocks were prepared from IMWITOR® 900 K (solid wax), IMWITOR® 372 P (solid wax). Tween 80, Labrafil® M 1944, Labrasol®,. Concentrations of the non-active ingredients in the soft-SPNF are listed in Table 16.

TABLE 16
Soft SPNF-blank (MA-9-60-Stock)

| Material | % w/w |
|---|---|
| IMWITOR ® 900 K* | 10 |
| Tween 80 | 20 |
| Labrasol ® | 60 |
| Labrafil ® M 1944 CS | 10 |

*Can be replaced with IMWITOR 372 P with similar results.

For blank preparation, materials were weighed and heated while stirring to obtain a homogenous clear and transparent liquid mass. The mixture was cooled to room temperature. The soft SPNF had waxy appearance.

For soft SPNF-drug preparation, 200 mg of (1) Curcumin, (2) paclitaxel and (3) CBD were mixed with 1800 mg melted soft SPNF-blank (10% drug w/w) until complete solubilization. The drugs dissolve immediately. The mixture was cooled to room temperature.

### Results and Conclusions

Particle size analysis was performed with freshly prepared water dispersed soft SPNF (1), i.e., soft SPNF-Curcumin, and the respective blank by DLS. The particle size of the blank was 107.3±2.3 nm (PDI: 0.183±0.023) as opposed to the size of the soft SPNF-Curcumin of 164.0±1.1 nm (PDI: 0.178±0.024).

The shelf-life stability of the soft SPNF-Curcumin and blank were further evaluated after 2 months storage at room temperature by visual and particles size analyses. The results suggested that the soft SPNFs remain homogeneous with no traces of sedimentation or other changes of color and texture. The particle size of the dispersed formulations remained within the same range. Further studies on soft SPNFs are currently ongoing.

### Example 7: Effects of CBD Solid PNL Formulation In-Vitro and In-Vivo

The objective has been to prepare and characterize two PNL formulations, SPNF-CDB and LPNF-CBD absorbed into MCC powder (microcrystalline cellulose), in-vitro and in-vivo.

### Materials and Methods

The reagents included: Solutol HS 15, Cremophor RH 40, Tween 20, Tween 80, span 80, Propylene glycol, PEG 1000, Labrafil M1944, Sesame oil, Microcrystalline Cellulose (MCC), Imwitor 900 and CBD powder (PureForm Global).

Solutol HS 15 (also Koliphor HS 15) is Polyoxyl 15 Hydrostearate is a nonionic solubilizer and an emulsifying agent, approved by the FDA for parental and ophthalmic use. It is water soluble yellowish white paste at room temp with melting point around 30° C.

Cremophore RH 40 (also Koliphor RH 40) is PEG-40 hydrogenated castor oil. It is a non-ionic solubilizers and an emulsifying agent that is obtained by reacting hydrogenated castor oil with ethylene oxide.

IMWITOR 900 K is an ester of natural plant derived fatty acids (stearic acid and palmitic acid) and glycerol. It is white to light yellowish, solid, waxy powder, nearly neutral in odour and taste. It does not contain additives or residues of antioxidants, stabilizers, or solvents. It has a melting point around 61° C. It has high storage stability and no rancidity risk due to lack of unsaturated components. It has very good skin compatibility and can be used orally (classified as GRAS). It is used as lipophilic matrix for oral solid dosage forms (granulation, hot melt technique), tablet lubricant, emulsion stabilizer and dispersing agent for pigments and for sustained release.

Tween 80 (Polysorbate 80) is a nonionic surfactant and emulsifier often used in foods and cosmetics. It is water soluble viscous yellow liquid.

Tween 20 (Polysorbate 20) is a nonionic surfactant and emulsifier. It is water soluble viscous yellow liquid that used as an excipient in pharmaceutical applications to stabilize emulsions and suspensions.

Span 80 (Sorbitan Oleate) is a nonionic surfactant. It is light yellow viscous oily liquid that used as emulsifier and stabilizer in food and medicine.

Labrafil® M 1944 is Oleoyl polyoxyl-6 glycerides, a nonionic water-dispersible surfactant for lipid-based formulations to solubilize and increase oral bioavailability of poorly water-soluble actives. It is liquid at room temperature and self-emulsifies in aqueous media to form a coarse dispersion (emulsion, SEDDS). It is further used as a co-emulsifier in topical formulations to improve stability of emulsions.

Propylene glycol (propane-1,2-diol) is water soluble viscous, colourless liquid, with slightly sweet taste. It is considered a better general solvent than glycerine and dissolves a wide variety of compounds. It has become widely used as a solvent, extractant and preservative in a variety of parenteral pharmaceutical formulations, and further as a plasticizer in aqueous film-coating formulations, and as a carrier for emulsifiers in cosmetics and food industry.

For solid LPNF-CBD, Solutol HS 15, Cremophor RH 40, Propylene glycol, Span 80, Tween 20 and CBD were mixed in specified weight ratios, while shaking at 37° C. Powder was prepared by absorbing equal amounts of LPNF and MCC and mixing to ensure homogeneity. The ingredients and their respective proportions are listed in Table 17.

TABLE 17
Composition of LPNF-CBD

| Material | % w/w |
|---|---|
| Cremophor RH 40 | 13% |
| Tween 20 | 18% |
| Span 80 | 18% |
| Solutol HS 15 | 6% |
| Propylene glycol | 20% |
| Sesame oil | 10% |
| CBD | 5% |

For SPNF-CDB, Tween 80, Labrafil M1944, Cremophor RH 40, PEG 1000, Imwitor 900 and CBD were incubated in specified weight ratios at 60° C., while stirring to ensure homogeneity. The mixture of melted materials was cooled to room temperature for 1 h. The ingredients and their respective proportions are listed in Table 18.

TABLE 18
Composition of SPNF-CBD

| Material | % w/w |
|---|---|
| Tween 80 | 8% |
| Labrafil M1944 | 28% |
| Cremophor RH 40 | 8% |
| PEG 1000 | 8% |
| Imwitor 900K | 38% |
| CBD | 10% |

The ingredients of the liquid control formulation are listed in Table 19.

TABLE 19
Composition of the liquid control

| Material | % w/w |
|---|---|
| Ethyl lactate | 35 |
| Lecithin | 7.2 |
| Tricaprin | 13.2 |
| Tween 20 | 13.2 |
| Span 80 | 13.2 |
| Cremophor RH 40 | 13.2 |
| CBD | 5 |

For the animal study, water suspensions of the two formulations were prepared by suspending 500 mg SPNF-CDB and 1 g LPNF-CBD powder in 10 ml DDW to achieve the effective concentration of 5 mg CBD per ml.

For animal studies, male Wistar rats (0.295-0.335 kg) were tested in three groups: (1) receiving the LPNF-CBD powder, (2) SPNF-CDB and (3) liquid control formulation (CBD dose 15 mg/kg per oral administration in all groups. The formulations were administered to the animals by oral gavage. Systemic blood samples (0.35 mL) were taken at pre-dose (5 min) and at specific post-dose time points (0.3, 0.6, 1, 1.5, 2, 4, 6 and 8 h). Plasma was separated by centrifugation (4000 rpm, 10 min) and stored at −20° C. pending analysis.

Plasma aliquots (150 μL) were spiked (10 μL) an with internal standard, Cannabigerol (CBG 1 μg/mL). Acetonitrile (ACN 150 μL) was added and mixed for 2 min. CBD/CBG extraction was performed by N-hexane (3 mL) added to test tubes (A), followed by mixing for 2 min. After centrifugation at 4000 rpm for 10 min, the N-hexane organic layer was transferred to fresh glass test tubes (B) and evaporated to dryness (Vacuum Evaporation System, Labconco,). Tubes B were reconstituted in 80 μL of ACN:water (80:20). The resulting solution (80 μl) was injected into the HPLC-MS-MS system (Waters pump (600 controller), Waters autosampler (717 plus Auto-sampler) and Waters Micro-mass ZQ mass spectrometer (Waters corporation, Milford)).

### Results and Conclusions

In terms of appearance and behavior in suspension, solid LPNF-CBD was a white dry powder that dispersed immediately in aqueous solution. SPNF-CBD had a solid waxy texture with a melting point at about 55° C. It dispersed easily (few minutes) in aqueous solution, yielding a milky suspension. Particle size and PDI characteristics of water suspensions of the two test formulations and control are detailed in Table 20.

TABLE 20
Particle size and PDI in water suspensions
of the tested formulations and control

| | | CBD | | |
|---|---|---|---|---|
| | | loading | | |
| Sample code | Appearance | (w/w) | Size (nm) | PDI |
| MAE-3-129-A | SPNF | 10% | 141 ± 1.301 | 0.296 ± 0.064 |
| MAE-3-129-B | Powder | 5% | 34.45 ± 0.606 | 0.279 ± 0.003 |
| | LPNF | | | |
| Control | Liquid | 5% | 26.02 ± 0.7 | 0.136 ± 0.01 |

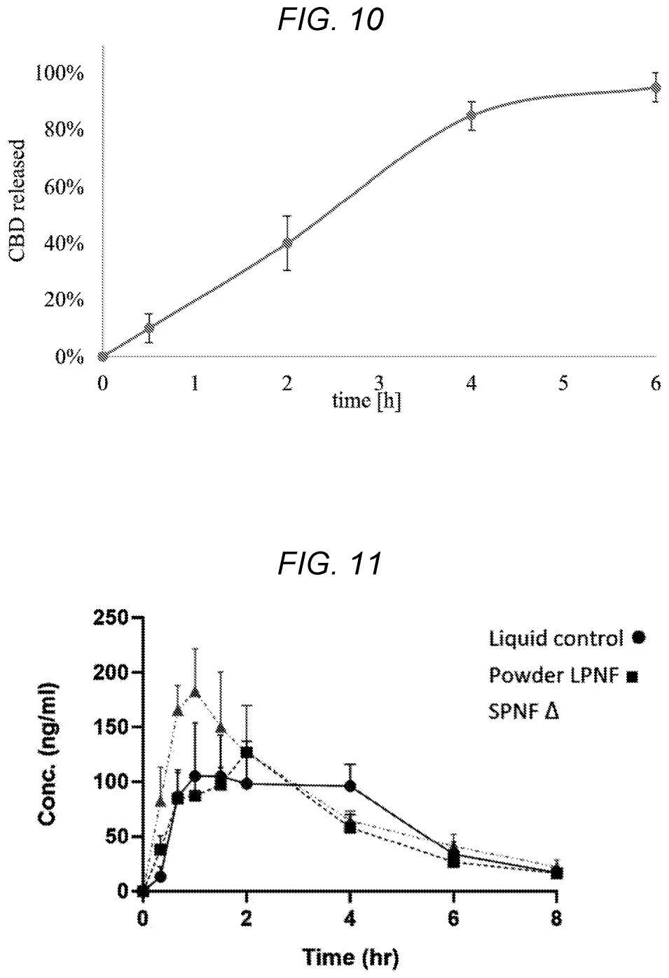

In suspension, both types of solid formulations, powder LPNF and SPNF, produced particles with an average size of <200 nm, and specifically of about 34 nm and about 140 nm, respectively. Studies in vitro of the CBD release from the powder LPNF in GI mimicking conditions (gastric solution (pH˜2) for 2 h and intestinal solution (pH˜7) for additional 2-6 h), showed a prolonged release profile, with increasing release of CBD for up to 4 h and reaching plateau after 5-6 h. These results are shown in FIG. 10.

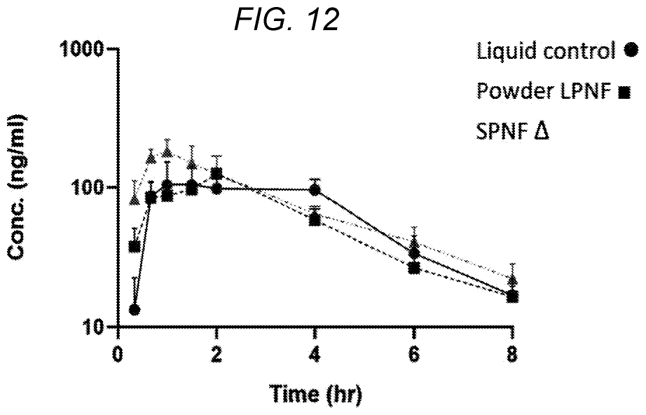

Pharmacokinetic (PK) studies used water suspensions of the three formulations (X10), administered per os to rats in equal effective doses (15 mg CBD/kg). The crude data is shown in FIG. 11 and in semilogarithmic scale in FIG. 12. Subsequent more detailed analysis of the PK parameters is shown in Table 21, suggesting that the SPNF-CBD formulation had the best PK performance in terms of AUC, Cmax and Absolute bioavailability.

TABLE 21
Analysis of PK parameters in the animal study

| | Control * | Powder | |
|---|---|---|---|
| | formulation | LPNF-CBD | SPNF-CBD |
| Mean ± SD | (n = 4) | (n = 7) | (n = 6) |
| AUC₀₋₁₀ (ng*hr/ml) | 545 ± 160 | 485 ± 162 | 633 ± 221 |
| AUC_{0-∞} #(ng*hr/ml)** | 565 ± 156 | 527 ± 172 | 724 ± 221 |
| Cmax (ng/ml) | 150 ± 58.1 | 140 ± 31 | 212 ± 101 |
| K (terminal slope 1/hr) | 0.43 ± 0.02 | 0.33 ± 0.13 | 0.32 ± 0.13 |
| T_{0.5} (hr) | 1.61 ± 0.07 | 2.36 ± 0.79 | 2.53 ± 1.16 |
| F_{abs} (% Absolute | 12.5 ± 3.45 | 11.70 ± 3.82 | 16.06 ± 4.91 |
| bioavailability)*** | | | |

*Result for the liquid control formulation were taken from previous results.
**# AUC0-∞ was calculated for infinity using the terminal slope.
**Fabs (% of Absolute bioavailability) was calculated relative to the liquid control formulation administered by IV.

### Annex A

TABLE 17
Pain related conditions that can be treated with CBD or CBD:THC combined

| Cannabinoid | Medical Condition | Reference |
|---|---|---|
| CBD | Unremitting pain in RA | |
| | MS pain, spinal cord injury, brachial | Wade et al. 2003 |
| | plexus injury, limb amputation | |
| | Chronic pain associated with kidney | Cunetti et al. 2018; Xu et al. 2020 |
| | transplantation (peripheral neuropathy) | |
| | Neuropathic pain | Notcutt et al. 2004 |
| | Opioid use and quality of life and sleep in | Capano, Weaver, and Burkman 2020 |
| | patients with chronic pain | |
| | Anxiety, depression and psychotic | Garcia-Gutierrez et al. 2020 |
| | disorders | |
| | Virus infections including Covid-19 | Anil et al. 2021; Nguyen et al. 2022 |
| CBD:THC | Central pain in MS | Rog et al. 2005; Rog, Nurmikko, and |
| combined | | Young 2007; Russo et al. 2016; van de |
| | | Donk et al. 2019 |
| | Intractable cancer pain | Johnson et al. 2010; Portenoy et al. 2012 |
| | Neuropathic pain following spinal cord | Mondello et al. 2018 |
| | surgery | |
| | Peripheral neuropathic pain | Donvito et al. 2018; Fine and Rosenfeld |
| | | 2013; Hoggart et al. 2015; Serpell et al. |
| | | 2014 |
| | RA pain | Blake et al. 2006 |
| | Chemotherapy induced neuropathic pain | Lynch, Cesar-Rittenberg, and Hohmann |
| | | 2014 |
| | Rheumatic diseases (Fibromyalgia, back | Fitzcharles et al. 2016 |
| | pain, OA, RA) | |
| | Fibromyalgia | Berger et al. 2020; van de Donk et al. |
| | | 2019 |
| | IBD | Gotfried, Naftali, and Schey 2020; |
| | | Naftali et al. 2014 |
| | Sleep quality | Russo, Guy, and Robson 2007 |
| | Autism | Aran et al. 2021 |
| | Pain in Alzheimer's Disease | Uddin et al. 2020 |

1.-42. (canceled)

43. A solid or semi-solid composition comprising at least one lipophilic active and a solid or semi-solid mixture comprising at least one material from the following groups:
a surfactant or an emulsifier,
a lipid selected from the group of fatty acids, fatty acid esters, fatty amines, fatty amides and fatty alcohols,
an amphiphilic solvent,
and further optionally comprises at least one absorbent,
wherein the composition has a melting point at a temperature of at least about 30° C., and
wherein upon contact with an aqueous medium, the composition provides a controlled release and improved bioavailability of the at least one lipophilic active.

44. The composition of claim 43, wherein the lipid is selected from the group of mono-, di- and triglycerides, fatty alcohols and waxes.

45. The composition of claim 43, wherein the surfactant or emulsifier is selected from the group of Tweens, Spans, Labrasoles, Labrafils.

46. The composition of claim 43, wherein the amphiphilic solvent is selected from the group of PEG 200-400, ethanol, isopropanol, propylene glycol, glycerol, ethyl lactate, ethyl acetate.

47. The composition of claim 43, wherein the at least one adsorbent is selected from the group of microcrystalline celluloses, chemically modified celluloses or starch, polyacrylates, chalk, charcoal, porous silica and synthetic allyl polymers.

48. The composition of claim 47, wherein the polyacrylates are selected from the group of copolymers of acrylic acid and methyl methacrylate.

49. The composition of claim 47, wherein the synthetic allyl polymer is Polypore.

50. The composition of claim 47, wherein the at least one adsorbent is microcrystalline cellulose.

51. The composition of claim 43, wherein the at least one lipophilic active is selected from the group of lipophilic therapeutic and nutraceutical agents.

52. An ophthalmic formulation, a topical formulation, a cosmetic formulation, a food, a food supplement, or a food additive comprising the composition of claim 43.

53. The composition of claim 43 for improved delivery of lipophilic actives to a mammal.

54. A method of treating a disorder or a condition in a mammal, the method comprises administering to the mammal the composition of claim 43.

55. The method of claim 54, wherein the mammal is human.

56. The method of claim 54, wherein the disorder or the condition is treatable by cannabinoids or combination thereof.

57. The method of claim 54, wherein said administering comprises oral, buccal, ophthalmic, topical delivery or subcutaneous, intramuscular, intravenous and intraocular injections, or inhalation of the composition to the mammal.
